(19) United States
(12) Patent Application Publication
(10) Pub. No.: US 2019/0262489 A1
(43) Pub. Date: Aug. 29, 2019

(54) PLASMA STERILIZATION AND FEEDBACK SYSTEM

(71) Applicant: PLASMAMEDIC LTD., Netanya (IL)

(72) Inventors: Yigal YANAI, Givataym (IL); Jacques ZYLBERG, Givat Shmuel (IL)

(21) Appl. No.: 16/305,572

(22) PCT Filed: May 30, 2017
(86) PCT No.: PCT/IL2017/050606

§ 371 Date: Nov. 29, 2018

Related U.S. Application Data
(60) Provisional application No. 62/343,157, filed on May 31, 2016.

(51) Int. Cl. A61L 2/28; A61L 2/24; A61L 2/14

(52) U.S. Cl. CPC A61L 2/28; A61L 2/24; A61L 2202/11; A61L 2202/14; A61L 2/14

(57) ABSTRACT

A plasma sterilizer including a plasma driver configured to provide plasma to a target area, a dispenser configured to apply an indicative substance to the target area, a light detector configured to detect an integrated luminescence intensity of the indicative substance from the target area, and a feedback processing unit configured to obtain information indicative of the emitted luminescence from the light detector and to control the operation of the plasma driver

### TECHNICAL FIELD

The present disclosure generally relates to the field of plasma based sterilization and evaluation of sterilization process and efficiency.

### BACKGROUND

Sterilization is essential for most medical processes and procedures and involves the elimination of microbial life and other disease-causing pathogens from medical devices, surfaces, skin surfaces and open wounds or surgical incisions. Many medical products are delicate and cannot undergo sterilization by heat or aggressive chemical agents.

Plasma sterilization is a relatively new technology providing non-toxic sterilization at room temperature. The sterilization is primarily achieved through the the interaction of advanced oxidizing products (AOPs) such as radicals and ions and other reactive oxygen and nitrogen species (RONS) as well as by an etching effect by the acceleration of charged particles within a strong electrical field with components of the microorganism thereby causing their extermination.

Plasma sterilization is a very efficient method for eradicating pathogens, however the interaction between the plasma components and the various different surfaces encountered and their non-linear and curved nature presents challenges to the use of plasma systems without the aid of a real time feedback mechanism. There is, therefore, a need for a feedback mechanism and/or processing system capable of quantitatively and qualitatively evaluating the sterilization process in real time, thus enabling modifying its intensity and components to ensure achieving the desired results.

### SUMMARY

The present disclosure relates to methods and apparatus for using a plasma based sterilizer and systems containing the sterilizer, wherein the sterilizer includes a feedback processing system enabling real-time evaluation of the sterilization process. The feedback processing system is based on the fact that the plasma is capable of changing a property of an indicative substance applied to the target object. For example, the indicative substance may be a luminescent material, such as, for example, a fluorescent material; and the plasma may induce a change in the wavelength of the emitted fluorescence.

Advantageously, the change in the property of the indicative substance is correlative to the extermination and/or inactivation of the pathogen. That is, the amount of plasma (plasma flux) required to induce the change in the indicative substance (such as a change in the wavelength of the emitted luminescence) corresponds to the amount of plasma (plasma flux) required for extermination and/or inactivation of the pathogen. As a result, the change in the property of the indicative substance may serve as a reliable and continuous indication of the sterilization process. Accordingly, if a parameter affecting the plasma treatment is altered, such that the intensity and/or efficiency of the treatment is affected, the change in the property of the indicative substance is affected correspondingly. For example, if the ambient humidity changes the concentration of species in the plasma, reducing the efficiency of the plasma sterilization process, then the time required to bring about the change in the wavelength of the luminescence emitted by the indicative substance is increased. This ensures that proper sterilization is achieved.

The continuous input from the indicative substance is used as an input commanding signal to the plasma generating system to increase, reduce or stop altogether its operation according to the actual disinfection results achieved, in real time. Inputs from other environmental parameters such as temperature and humidity sensors may also be integrated. As plasma sterilization frequently encounters highly curved non-linear surfaces, a range finder may also be integrated in order to ensure even exposure to the plasma, regardless of the position of the sterilized area relative to the plasma source.

An additional advantage of the feedback processing system, disclosed herein, is that the measured parameter, i.e. a wavelength of luminescence emitted by the indicative substance as a result of its interaction with the plasma, may be selected so as to be unique to the indicative substance. This avoids the main drawback of evaluations based on a direct measure of components obtained as a result of decomposition of the microorganism, namely inaccuracies resulting from components desorbed from the object, subject to sterilization, or from the surroundings. For example, evaluations based on measuring desorption of carbon, oxygen or other organic component from the microorganism, as a result of its decomposition, may be distorted by the desorption of such components from the sterilized object itself. Similarly, evaluations based on measuring hydrogen desorbed from the decomposing microorganism may be distorted by water or water containing liquids on the target or its surroundings.

A problem often encountered during plasma sterilization is unequal penetration of the treatment, especially when the treatment object is non-linear surfaces, such as the hands or an open surgical cut. Using the feedback processing system disclosed herein ensures that such complex targets/surfaces receive an evenly distributed exposure to plasma treatment by increased or prolonged treatment in required regions identified by study of the selected property of the indicative substance and is only terminated when a sufficient change in the property of the applied indicative substance is accomplished over the entire target area. That is, after the application of the luminescent indicative substance to the target object or surface, the luminescence of the object/surface may be detected, thereby ensuring complete coverage by the indicative substance. Once complete coverage is assured, the plasma treatment may be commenced and the efficiency of the sterilization along all surfaces may be evaluated based on the measurement of the changed luminescence of the indicative substance.

According to some embodiments, there is provided a plasma sterilizer comprising: a plasma driver configured to provide plasma to a target area, a dispenser configured to apply an indicative substance to the target area, a light detector configured to detect an integrated luminescence intensity of the indicative substance from the target area; and a feedback processing unit configured to obtain from the light detector information indicative of the emitted luminescence and to control the operation of the plasma driver based at least on the information. An example would be to control the operation of one or more operational functions of the plasma driver based on an analysis of the luminescence emitted from the target area, wherein the luminescence emitted from the target area is indicative to the level of decontamination of the target pathogen(s).

According to some embodiments, there is provided a method for plasma sterilizing, the method comprising: applying an indicative substance to a target area; utilizing a plasma driver, providing plasma to the target area; detecting an integrated luminescence intensity of the indicative substance from the target area; utilizing a feedback processing unit, obtaining information indicative of the luminescence emitted from the target area and controlling one or more operational functions of the plasma driver based on an analysis of the luminescence emitted from the target area, wherein the luminescence emitted from the target area is indicative of the level of decontamination of target pathogen(s) on (and/or in) the target area.

According to some embodiments, the target area may be divided into pixel or pixel-like sub-areas; each pixel or pixel-like sub-area may be analyzed (e.g., for returned luminescence) and/or controlled individually (e.g., operation of the plasma bombardment to this particular sub-area). The analysis and plasma treatment operation of each sub-area may be automatically tracked and/or operated.

According to some embodiments, there is provided a method for plasma sterilizing, the method comprising: applying an indicative substance to the targeted area, wherein the indicative substance comprises a “good bacteria” bound to or otherwise marked with a luminescence emitting substance, wherein the luminescence emitting substance is configured to emit luminescence only when it is bound to the “good bacteria” and/or only when the “good bacteria” is intact; detecting an integrated intensity of the luminescence over the targeted area using a detector; treating the targeted area with plasma using a plasma sterilizer, thereby inducing a change in the indicative substance; and detecting the integrated intensity of the luminescence over the targeted area, thereby determining the level of sterilization of the target area. The change in the indicative substance may include damage caused to the “good bacteria” and, as a result, the luminescence decreases below a threshold level or is terminated.

According to some embodiments, the method may further include continuing or repeating the step of treating the targeted area with plasma until the integrated intensity of the luminescence over the targeted area has decreased below a threshold level.

According to some embodiments, a termination of luminescence or a decrease thereof below a threshold level is indicative to substantial damage to (destruction of) pathogens in the target area.

According to some embodiments, the plasma sterilizer further comprises a light source configured to provide light at a wavelength capable of inducing luminescence by the indicative substance.

According to some embodiments, the plasma sterilizer further comprises a gas blender.

According to some embodiments, controlling the operation of the plasma driver comprises controlling current, frequency, voltage, timing and modulation or any combination thereof.

According to some embodiments, the plasma sterilizer further comprises at least one gas supply input configured to provide the plasma generator with gas composition, wherein the gas composition is determined by the feedback processing unit and/or by manual input.

According to some embodiments, the plasma sterilizer further comprises one or more sensors configured to provide sensor signals to the feedback processing unit, wherein the feedback processing unit is configured to determine the gas composition and to determine the output setting of the plasma power driver based on the sensor electric signals.

According to some embodiments, the sensor signals comprise: in-flow gas composition, distance between the plasma generator and the target area, volume of confined space, ambient temperature, temperature on target surface, ambient humidity, current leakage from the plasma to the target, specific particle concentration or any combination thereof. According to some embodiments, the specific particle comprises, NxOy or ozone.

According to some embodiments, the light detector is configured to detect a wavelength of the emitted luminescence from the target area. According to some embodiments, the feedback processing unit is further configured to associate the wavelength and/or integrated intensity of the emitted luminescence from the targeted area, with an amount of plasma delivered to the target area and/or with a sterilization level achieved. According to some embodiments, the light detector is a camera.

According to some embodiments, the indicative substance emits luminescence at a first wavelength before interaction with the plasma provided by the plasma generator and emits luminescence at a second wavelength after interaction with the plasma provided by the plasma generator. According to some embodiments, the light detector is configured to differentiate between the first and the second wavelength and/or associated integrated intensity. According to some embodiments, the feedback processing unit is configured to control the level and mode of activation of the plasma power driver and/or gas blender based on a ratio between the first and the second wavelengths and/or associated integrated intensity.

According to some embodiments, the light source is a UV light source.

According to some embodiments, the luminescence comprises fluorescence, phosphorescence or both.

According to some embodiments, the indicative substance is able to selectively bind and/or be absorbed by microorganisms.

According to some embodiments, the plasma sterilizer further comprises at least one gas supply input, wherein the opening and/or closing and/or flow control of the gas supply input is controlled by the feedback processing unit. According to some embodiments, the at least one gas supply input comprises at least two gas supply inputs, wherein the opening and/or closing and/or flow control of each of the gas supply inputs is separately controlled by the feedback processing unit.

According to some embodiments, the plasma sterilizer further comprises a graphic and/or numerical user interface configured to display the intensity of the emitted luminescence, the amount of plasma delivered to the target area, the sterilization efficiency, the sterilization progress, the level of disinfection obtained or any combination thereof.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1A shows an exemplary graph illustrating the correlation between intensity of luminescence of an indicative substance and pathogen extermination and/or inactivation as a result of plasma treatment;

FIG. 1B shows an exemplary graph illustrating the correlation between intensity of luminescence of an indicative substance and pathogen extermination and/or inactivation as a result of plasma treatment;

FIG. 2 schematically illustrates a plasma sterilizing system, according to some embodiments;

FIG. 3 schematically illustrates a method for evaluating plasma sterilization efficiency, according to some embodiments;

FIG. 4 is an exemplary flowchart for the operation of a plasma sterilization feedback processing system, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a plasma sterilizer including a plasma generator configured to provide plasma, an indicative substance dispenser configured to apply an indicative substance to a target area, a light source configured to provide light at a wavelength capable of inducing luminescence from the indicative substance, a detector configured to detect the emitted luminescence; and upon receiving the input from the indicative substance, the plasma driver shall vary the intensity and/or the components of the plasma emitted. This variation can be on the basis of a pre-programmed desired protocol as well as a result of a manual or another input command that would result in changing the plasma and/or AOP components.

According to some embodiments, the plasma sterilizer may be a cold plasma sterilizer. According to some embodiments, the plasma sterilizer may be hand held. According to some embodiments, the plasma sterilizer may be suitable for sterilization of skin surfaces such as, for example, the hands of a subject. According to some embodiments, the plasma sterilizer may be configured for attachment to patient beds, to entrances into rooms (such as, but not limited to, operation rooms) or to any other suitable location. As a non-limiting example, the plasma sterilizer may be attached at the entrance to patient rooms in a hospital, thereby enabling medical personnel to sterilize their hands before and/after approaching a patient. According to some embodiments, the plasma sterilizer may include a plurality of nozzles through which the plasma treatment is provided. According to some embodiments, the plasma sterilizer may include an array of nozzles through which the plasma may be generated. According to some embodiments, each of the nozzles in the nozzle array and/or sub-segments of the array may be separately controllable. According to some embodiments, each of the nozzles in the array and/or sub-segments of arrays may be separately connected to a gas provider and may be driven by sub-segment specific power settings. Accordingly, each nozzle in the array and/or each sub-segment of the array may provide plasma made from a same or a different mixture of gasses and at a same/or different timing. According to some embodiments, the plasma array may be configured to enable contouring of the treatment area based on the identification of areas, which require additional sterilization. For example, the array may utilize one or more nozzles (e.g. 2, 3, 4, 5, 10 or more) and/or sub-segments of the array through which plasma treatment is selectively provided, so as to direct the plasma treatment specifically to areas requiring additional treatment. According to some embodiments, the plasma array may be configured to enable contouring of the treatment area based on the identification of areas, which require enhanced sterilization. For example, the array may utilize one or more nozzles (e.g. 2, 3, 4, 5, 10 or more) and/or sub-segments of the array through which plasma treatment is selectively provided, so as to direct the plasma treatment specifically to areas a priori identified as requiring a more intense treatment (e.g. areas with intense contamination and/or areas difficult to access such as between fingers).

According to some embodiments, there is provided a plasma sterilization feedback processing system, the processing system including an indicative substance dispenser configured to apply an indicative substance to a target area, a light source configured to provide light at a wavelength capable of inducing luminescence from the indicative substance; a detector configured to detect the emitted luminescence; and a software controlled plasma driver configured to drive a plasma generator based on the detected luminescence.

As used herein the term “plasma” refers to an ionized gas. Plasma is one of the four fundamental states of matter, the others being solid, liquid, and gas. According to some embodiments, the plasma may be cold plasma.

A plasma can be created by heating a gas or subjecting it to a strong electromagnetic field applied with a laser, a microwave generator or electrical discharge so as to induce electronic and chemical processes in which molecules may be elevated to electronically excited metastable states and/or decompose into neutral radical and/or ions. These reactive species may then react further with their surrounding compounds.

As used herein, the terms “plasma sterilization” and “plasma treatment” may be used interchangeably and refer to the process of applying plasma to a target area in order to eliminate microorganisms and other pathogens therefrom. Without being bound by any theory, plasma sterilization includes the generation of reactive species such as reactive oxygen/nitrogen species (RONS) capable of interacting with essential components of various kinds of pathogens, thereby leading to their destruction. In addition, plasma sterilization operates by exploiting an etching effect coming into play by the acceleration of charged particles within a strong electrical field.

As used herein the terms “luminescence indicative substance”, “indicative substance”, “luminescent substrate” “labeling substance” and “luminescent label” may be used interchangeably and may refer to any substance configured to emit luminescence. According to some embodiments, the labeling substance may be configured to emit luminescence at a first wavelength prior to the interaction with plasma and to emit luminescence at a second and optically distinct wavelength or lose its emittance capabilities altogether after the plasma treatment. Additionally or alternatively, the labeling substance may be configured to emit luminescence at a first intensity prior to the interaction with plasma and to emit luminescence at a second intensity after the plasma treatment. According to some embodiments, the labeling substance may be configured to emit luminescence prior to the plasma treatment and to cease or significantly reduce the luminescence as a result of the treatment. According to some embodiments, the change in the luminescence of the labeling substance due to the plasma treatment may be correlated to the extermination and/or inactivation of the pathogens, as a result of the treatment. According to some embodiments, the correlation may be linear. According to some embodiments, the correlation may be non-linear. According to some embodiments, the labeling substance may be configured to specifically and/or selectively bind and/or be absorbed by microorganisms. According to some embodiments, the indicative substance may be a florescent material. Non-limiting examples of suitable indicative substances include: indocyanine green, fluorescein sodium and methylene blue or any combination thereof. Each possibility is a separate embodiment. Additionally or alternatively, the labeling substance may be a phosphorescent material.

According to some embodiments, the indicative substance may be configured to emit luminescence as a result of binding to an entity specific to the pathogen. That is, the indicative substance may be a substance which only emits luminescence as a result of the binding, also referred to herein as “binding induced luminescence” such that the intensity of the luminescence is correlative to the concentration of the bacteria. It is understood that the plasma treatment intensity and/or duration may be predetermined based on the intensity of the binding induced luminescence.

According to some embodiments, the indicative substance may include a “good/friendly bacteria” bound to or otherwise marked with a luminescence emitting substance. The luminescence emitting substance is configured to emit luminescence only when it is bound to the good/friendly bacteria and/or only when the good/friendly bacteria is intact. Once the plasma treatment causes damage to the good/friendly bacteria, the luminescence emission stops (for example, because the binding to the luminescence emitting substance is broken). Therefore, the intensity of the luminescence is correlative to the damage caused to the good/friendly bacteria as a result of the plasma treatment. Once the luminescence stops, this can be considered as an indication that the good/friendly bacteria are destroyed.

According to some embodiments, the good/friendly bacteria are pre-selected such that damage caused to them by the plasma treatment will necessarily cause damage to the pathogens, which such treatment is designed to destroy.

It is understood that the plasma treatment intensity and/or duration may be predetermined based on the intensity of the luminescence.

According to some embodiments, the good/friendly bacteria may be selected from the group consisting of: Lactobacillus acidophilus, Lactobacillus bulgaricus, Streptococcus thermophilous, Bifodophilus longum, Bifidobacteria bifidus, Bacillus laterosporus, Bacillus Bifidum, Lactobacillus plantaterum, Lactobacillus Rueteri, and Lactobacillus salivarus.

According to some embodiments, the good/friendly bacteria may also be referred to as probiotics.

According to some embodiments, genetically modified friendly bacteria may be used as fluorescent bio sensors (as an example of an indicative substance). According to some embodiments, nano-technology size fluorescent molecules (fluorescent nano-particles) may be used as sensors (as an example of an indicative substance). According to some embodiments, in both cases the sensors may be selected so that they represent a similar resistance and response curve to the plasma bombardment. In other words, to enable a representative feedback response from these markers (sensors), which will be indicative of the destruction curve of the real pathogens, the selected markers show a destruction curve similar to the real targeted pathogens under similar conditions. For example, if the target pathogens require X plasma intensity for Y duration of time in order to reduce their count by Z percentage, the same characteristics will be required for the selected markers.

Since each one of the pathogens may have different response characteristics in response to the bombardment of plasma, the correlation between the pathogens' response and the markers' response may be calculated/determined by laboratory experiments or by theoretical calculations.

As used herein, the terms “target area”, “target object” and “object of sterilization” and “object” may be used interchangeably and may refer to the surface subject to plasma sterilization treatment. Non-limiting examples of suitable objects/targets include, medical devices and equipment, bio-clean surfaces, bio-clean rooms, skin surfaces, open surgical incisions, implants, foodstuff (such as fruits and vegetables) and any other suitable objects/targets or combination thereof. Each possibility is a separate embodiment. Advantageously, materials and devices, which do not tolerate high temperatures and/or humidity, such as some plastics, electrical devices and corrosion-susceptible metal alloys, and fabrics, may be sterilized using the plasma treatment disclosed herein.

As used herein, the term “light source” may refer to any light source configured to induce luminescence of the indicative substance. According to some embodiments, the indicative substance may be configured to emit luminescence only when illuminated by the light source. According to some embodiments, the light source may be an integral part of the sterilizer. Additionally or alternatively, the light source may be a separate unit functionally connected to the sterilizer. According to some embodiments, the light source may emit UV light or black light. According to some embodiments, the light source may be a LED. According to some embodiments, the ambient light may be sufficient to detect the indicative substance.

As used herein, the term “detector configured to detect the emitted luminescence” may refer to any detector configured to detect the luminescence of the applied indicative substance. According to some embodiments, the detector may be configured to detect a wavelength of the emitted luminescence. According to some embodiments, the detector may be configured to differentiate between different wavelengths, such as between a first wavelength emitted by the indicative substance prior to its interaction with the plasma and a second wavelength emitted after the plasma treatment. Alternatively, the sterilizer may include more than one detector, such as a first detector configured to detect the uninduced luminescence of the indicative substance and a second detector configured to detect the luminescence emitted as a result of the plasma treatment. According to some embodiments, the detector may be an integral part of the sterilizer. Additionally or alternatively, the detector may be a separate unit functionally connected to the sterilizer. According some embodiments, the detector may be configured to determine an intensity of the emitted luminescence. According to some embodiments, determining the intensity of the emitted luminescence may include converting the intensity into a graduate scale, such as. but not limited to. a scale of 0-10, 0-100 or any other suitable scale which may be indicative of the intensity of the emitted luminescence. According to some embodiments, the detector may be an optical sensor such as a camera. According to some embodiments the detector may not be wavelength specific and detects emission within a range, limited by user or system setting or limited by device specifications. According to some embodiments the detector may scan a range of wavelengths outputting a wavelength dependent intensity diagram.

As used herein the term “control unit” may refer to a computer or any other processing device configured to control an output setting for the plasma driver/plasma generator based on the detected luminescence and optionally on information received from other sensors. According to some embodiments, the control unit may be configured to control the output setting for the plasma generator based on the wavelengths emitted by the indicative substance before, during and after the plasma treatment, respectively. According to some embodiments, the output setting for the plasma generator, controllable by the control unit may include in-flow gas composition, plasma driver setting, distance between the plasma generator and the target area, volume of the confined space when applicable, ambient temperature, ambient humidity, current leakage from the plasma to the target or any combination thereof. Each possibility is a separate embodiment. According to some embodiments, the plasma driver settings may include or refer to the operation of the power generator connected thereto, such as the current applied, voltage, frequency, modulation of signal, exposure duration, or any other suitable parameter or combination of parameters. Each possibility is a separate embodiment.

According to some embodiments, the control unit may be an integral part of the sterilizer. Alternatively, the control unit or parts thereof may be a separate unit functionally connected to the sterilizer. As a non-limiting example, the sterilizer may include an integral processing unit configured to receive the detected luminescence from the detector and to transfer, optionally wirelessly, the detected luminescence to an external processing unit configured to control the output settings for the plasma generator based on the transferred data. It is understood that such separation of the control unit into two or more processing units, optionally spatially separate processing units, is within the scope of the term “control unit”, as used herein.

According to some embodiments, the control unit may be configured to associate the wavelength and/or the intensity of the emitted luminescence, with an amount of plasma delivered to the target area and/or with a sterilization efficiency. According to some embodiments, the control unit may be configured to determine the amount of plasma required to induce the change in the property of the indicative substance, e.g. a change in the wavelength of the emitted luminescence and/or its intensity in order to ensure extermination and/or inactivation of the pathogens. According to some embodiments, determining the amount of plasma required to induce the change in the indicative substance, and thus to ensure extermination and/or inactivation of the pathogens, may include determining the intensity of the plasma treatment, the composition of the plasma, the duration of the plasma treatment, the repeated application of the plasma treatment under identical conditions, and/or the repeated application of the plasma treatment under a different set of parameters and/or any other parameter influencing the output of the plasma treatment. Each possibility is a separate embodiment. According to some embodiments, the control unit may be configured to determine the time required and/or time remaining for achieving a satisfactory sterilization. The operation of the plasma sterilizer, although being monitored by the control unit, may be overridden and controlled, online, manually by a user.

According to some embodiments, the plasma sterilizer and/or the control unit may be hand held. According to some embodiments, the plasma sterilizer and/or the control unit may be configured for attachment to patient beds, to entrances into room (such as, but not limited to, operating rooms) or to any other suitable location. As a non-limiting example, the plasma sterilizer, including the feedback processing system, may be attached at the entrance to patient rooms at a hospital, thereby enabling medical personnel, visitors or other users to sterilize their hands before and/or after approaching a patient. According to some embodiments, the feedback processing system may provide an indication to the user of when the sterilization process has terminated based on the change in the indicative substance, as described herein. According to some embodiments, the feedback processing system may operate in a closed loop with the plasma sterilizer. According to some embodiments, the feedback processing system may be configured to terminate the operation of the plasma sterilizer once complete sterilization has been achieved. According to some embodiments, the feedback processing system may be configured to adjust the operating parameters of the plasma sterilizer based on the change in the indicative substance. According to some embodiments, the feedback processing system may be configured to select specific nozzles through which plasma is provided, based on the change in the indicative substance. According to some embodiments, the feedback processing system may identify areas, which require additional sterilization and specify the operation of the plasma sterilizer specifically to those areas. For example, the feedback processing system may select one or more nozzles (e.g. 2, 3, 4, 5, 10 or more) from a plurality of nozzles (e.g. 10 or more, 20 or more, 50 or more 100 or more nozzles) through which plasma treatment is selectively provided, thereby directing the plasma treatment specifically to areas where sterilization is not complete. According to some embodiments, the feedback processing system may be configured to control the mixture of gasses utilized for the plasma provided to each nozzle in the array and/or to each sub-segment of the array. According to some embodiments, the feedback processing system may be configured to control the timing of the plasma treatment provided by each nozzle in the array and/or by each sub-segment of the array. According to some embodiments, the feedback processing system may be configured to enable contouring of the treatment area by identifying areas which require additional sterilization and or by identifying areas which require enhanced sterilization (e.g. between fingers).

According to some embodiments, the sterilizer may further include a display and/or graphic user interface configured to display the intensity of the emitted luminescence, the amount of plasma delivered to the target area, the sterilization level achieved, the time required/remaining to complete the sterilization process or any combination thereof. According to some embodiments, the display and/or graphic user interface may be an integral part of the sterilizer. Alternatively, the display and/or graphic user interface may be a separate unit functionally connected to the sterilizer. As a non-limiting example, the display and/or graphic user interface may be a display of a computer, a tablet, a smartphone, a website or any other suitable device configured to display the data to the user.

According to some embodiments, the sterilizer may further include at least one sensor (such as 1, 2, 3, 4, 5 or more sensors) configured to monitor an output parameter of the plasma treatment. The output parameters may be parameters influencing the efficiency or the processes of the plasma treatment. Non-limiting examples of suitable output parameters include distance between the plasma generator and the target area, temperature, within a confined space temperature at the target, ambient temperature, ambient humidity, current leakage from the plasma generator to the target area, specific particle concentration, or any combination thereof. Each possibility is a separate embodiment. As used herein, in accordance with some embodiments, the term “particle” may refer to a compound obtained as a result of the plasma treatment either as a result of desorption from the microorganism or from the treated object or as a byproduct of the treatment such as water, nitric oxides or emitted light. Each possibility is a separate embodiment. According to some embodiments, the data obtained from at least one sensor may be displayed on the display and/or graphic user interface. According to some embodiments, a control unit may be configured to adjust the output setting of the plasma driver/plasma generator/gas blender based on the monitored output parameter. As a non-limiting example, if the temperature of the plasma is determined to be too high, the control unit may be configured to control the operation of the plasma generator, thereby reducing the temperature of the plasma. As another non-limiting example, if the concentration of ozone, resulting from the treatment, is determined to be too high, the control unit may be configured to adjust the composition and/or blend of the input gases and/or the duration/modulation of the plasma treatment.

According to some embodiments, the sterilizer may further include at least one gas supply input. According to some embodiments, the opening and/or closing and/or flow control of the gas supply input may be controlled by the control unit. According to some embodiments, the sterilizer may include more than one gas supply input, such as, but not limited to 2, 3, 4, 5 or more gas supply inputs. Each possibility is a separate embodiment. According to some embodiments, the opening and/or closing and/or flow control of each gas supply input may be controlled separately by the control unit, thereby providing a real-time control over the blend of the gas supplied. According to some embodiments, the sterilizer may further include a plasma power driver functionally connected to the plasma generator. According to some embodiments, the operation of the plasma power driver, such as current, voltage, frequency, or any other suitable parameter or combination of parameters, may be controlled by the control unit. Each possibility is a separate embodiment.

According to some embodiments, the sterilizer may be mobile. As a non-limiting example, the sterilizer may include wheels enabling its movement along a surface. As another non-limiting example, the sterilizer may include suction arms or other mechanisms enabling it to crawl along surfaces, such as, but not limited to walls and ceilings. According to some embodiments, the sterilizer may be self-moving. According to some embodiments, the sterilizer may be mounted on or otherwise be attached to a robot. According to some embodiments, the control unit may include a navigation system configured to control the movement of the sterilizer. According to some embodiments, the control unit may be portable and/or hand-held.

According to some embodiments, there is provided a plasma sterilization feedback system, the system including an indicative substance dispenser configured to apply an indicative substance to a target area, a light source configured to provide light capable of inducing luminescence from the indicative substance; a detector configured to detect specific, one or more, types of luminescence or to detect a user defined range of emitted luminescence; combining all the input signals from the various sensors the control unit will process the data and determine the level and mode of activation of the plasma power driver and/or gas blender. According to some embodiments, a manual override will be possible.

According to some embodiments, there is provided a method for evaluating the plasma flux on the target, the method comprising applying an indicative substance to the targeted area, transmitting light to the targeted area, thereby inducing the indicative substance to emit luminescence; detecting the wavelength and/or integrated intensity of the luminescence over the targeted area using a detector; treating the targeted area with plasma, thereby inducing a change in the indicative substance, transmitting light from the light source, detecting the wavelength and/or integrated intensity of the luminescence over the targeted area, and determining the plasma flux over the targeted area based on the wavelength and/or integrated intensity of the detected luminescence, as essentially described herein.

According to some embodiments, there is provided a method for determining the level and mode of activation of the plasma power driver and/or gas blender, the method comprising applying an indicative substance on a targeted area, transmitting light to the targeted area, thereby inducing the indicative substance to emit luminescence; detecting the wavelength and/or integrated intensity of the luminescence using a detector; treating the targeted area with plasma, thereby inducing a change in the indicative substance, transmitting light from the light source, detecting the wavelength and/or integrated intensity of the luminescence over the targeted area, and determining the level and mode of activation of the plasma power driver and/or gas blender based on the wavelength and/or integrated intensity of the detected luminescence over the targeted area, as essentially described herein.

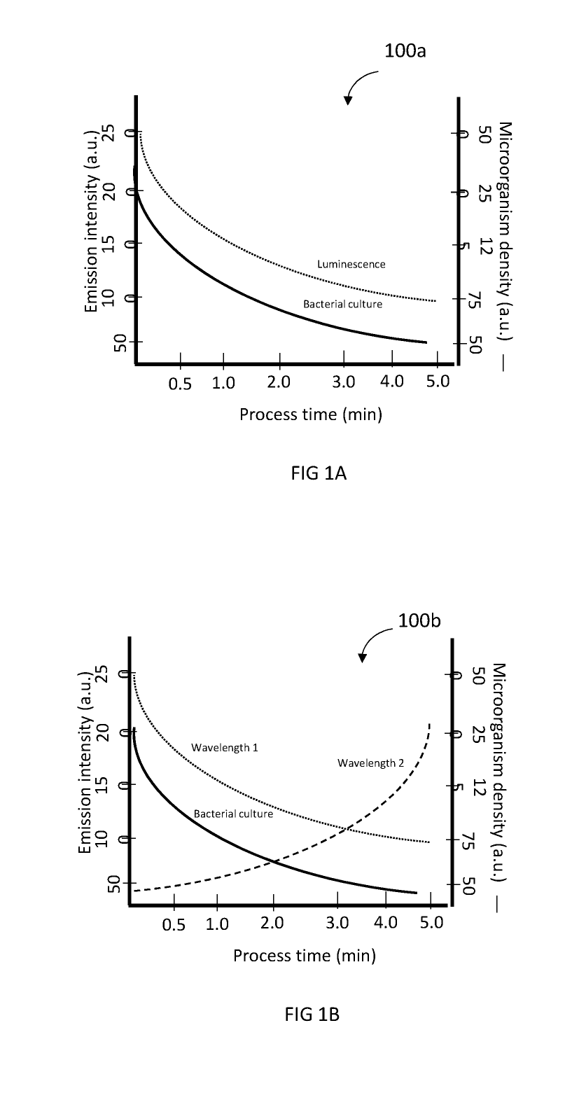

Reference is now made to FIG. 1A, which shows an exemplary graph 100a illustrating the correlation between intensity of luminescence of an indicative substance and microorganism extermination and/or inactivation as a result of plasma treatment. The indicative substance is, according to this embodiment, a material, which gradually loses it luminescence as a result of plasma treatment; however, other materials such as materials changing their luminescence or increasing their luminescence are also applicable and thus within the scope of the present disclosure. The intensity of luminescence during the treatment (dotted line) is determined using photometric analysis. The concentration of microorganisms (solid line), is determined based on a spectrophotometric analysis of bacterial cultures derived from samples obtained at time-points during the treatment. As seen from the figures there is a correlation between the extermination and/or inactivation of bacteria and the reduction in luminescence obtained from the indicative substance.

Reference is now made to FIG. 1B, which shows an exemplary graph 100b illustrating the correlation between intensity of luminescence of an indicative substance and microorganism extermination and/or inactivation as a result of plasma treatment. The indicative substance is, according to this embodiment, a material, which changes its luminescence as a result of the plasma treatment luminescence. That is, before the plasma treatment, the indicative substance emits light having a first wavelength (wavelength 1), whereas after the plasma treatment a second wavelength (wavelength 2) is emitted. The intensity of wavelength 1 luminescence (dotted line) and wavelength 2 luminescence (dashed line) over time of treatment is determined using photometric analysis. The concentration of microorganisms (full drawn line), is determined based on a spectrophotometric analysis of bacterial cultures derived from samples obtained at time-points during the treatment. As seen from the figure, there is a correlation between the extermination and/or inactivation of bacteria, the gradual disappearance of luminescence at wavelength 1 and the increase in wavelength 2 luminescence.

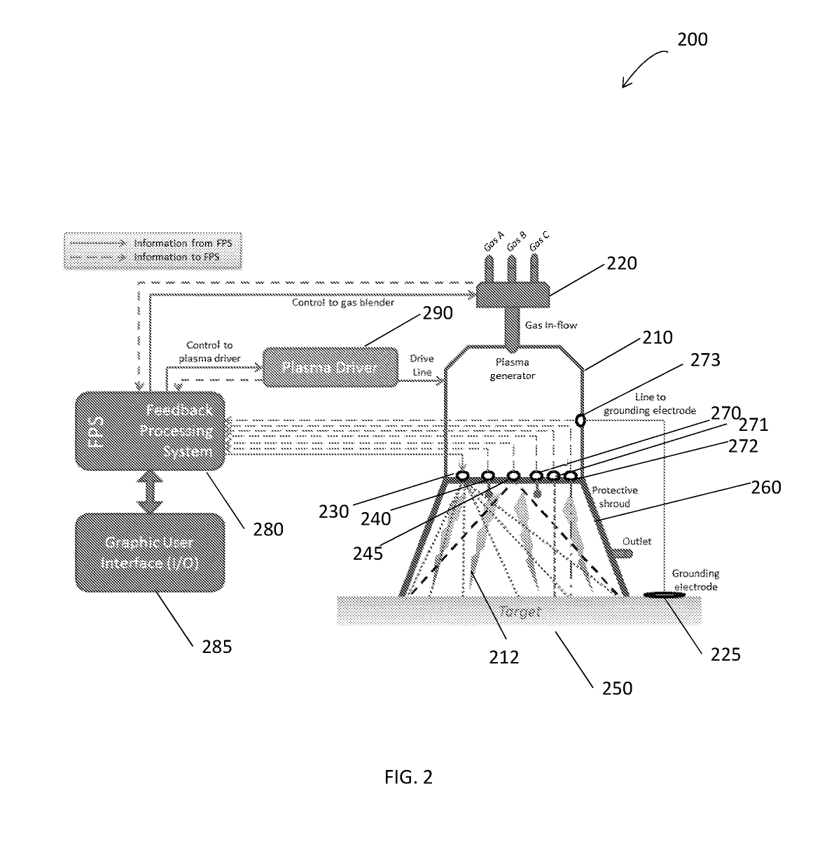

Reference is now made to FIG. 2, which schematically illustrates a plasma sterilizer system 200, according to some embodiments. Plasma sterilizer system 200 includes a plasma generator 210 configured to generate and/or provide plasma 212. Plasma generator 210 receives a gas (e.g. room air) or a gas blend (e.g. from one or more gas supplies) through gas supply input 220 and generates plasma by subjecting the inlet gas to an electric field generated by a power supply (not shown) and a ground electrode 225. However, other methods for generating plasma known in the art are also applicable and thus fall within the scope of the present disclosure. Plasma sterilizer system 200 also includes a dispenser 230 configured to apply an indicative substance to a target surface 250, a light source 240 configured to transmit light onto the indicative substance, thereby inducing its luminescence, and a light detector 245 configured to detect the intensity and/or wavelength of luminescence emitted by the indicative substance. Light detector 245 may detect the light emitted by the indicative substance before and after the treatment of target surface 250 by plasma 212, thereby enabling ongoing monitoring of the sterilization process, as essentially described herein. Plasma sterilizer system 200 may include a plasma directing structure 260 configured to direct plasma 212 to target 250 and to prevent its dispersion. Plasma directing structure 260 may also be configured to determine a fixed distance between plasma generator 210 and target 250. Optionally, plasma sterilizer system 200 may further include one or more additional sensors, such as, but not limited to, a humidity sensor, a thermometer, a current leakage detector, a distance meter or any other suitable sensor, here illustrated as sensors 270, 271, 272 and 273. Sensors 270, 271 and 272 may be configured to monitor one or more output parameter of the plasma provided, as essentially described herein. Plasma sterilizer system 200 further includes a feedback processing system 280. Feedback processing system (FPS) 280 may be formed integrally with plasma sterilizer system 200 or as a stand-alone processing unit functionally connected to plasma sterilizer system 200. Feedback processing system 280 is configured to receive data from light detector 245 and to control the output settings of a plasma driver 290 as well as the operation of dispenser 230 based on the received data, as essentially described herein. Feedback processing system 280 may further be configured to receive additional data from one or more sensors, such as sensors 270, 271 and 272 and to adjust the output settings of plasma driver 290 and/or plasma dispenser 230 based on the additional data, as essentially described herein. Feedback processing system 280 may further be configured to receive data from plasma driver 290 regarding currently used output settings, which data may be integrated into the determination of the preferred output settings and/or in the calculation of the sterilization efficiency. According to some embodiments, feedback processing system 280 may include a graphic user interphase 285 (input/output or I/O) configured to display the amount of plasma delivered to the target area, the sterilization efficiency, the time required/remaining to complete sterilization and/or any other data related to the sterilization process. According to some embodiments, the operation of plasma sterilizer system 200 may be fully or partially automated based on the operation of feedback processing system 280. Alternatively, the operation of plasma sterilizer system 200 may the manually controlled; in which case the operator may base his/her decisions, on the output settings of plasma driver 290 based on the output provided by feedback processing system 280. According to some embodiments, plasma sterilizer system 200 may be mobile, as essentially described herein.

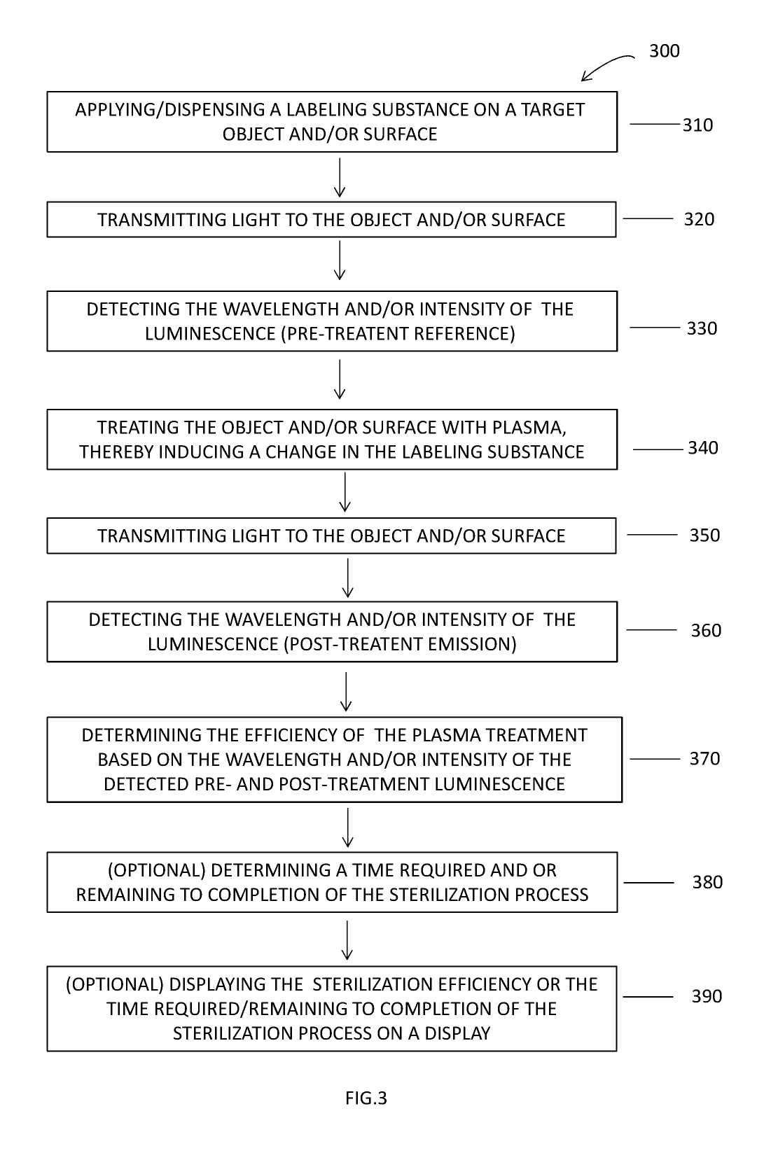

Reference is now made to FIG. 3, which is an exemplary flowchart of a method 300 for evaluating plasma sterilization efficiency, according to some embodiments. Step 310 of the method includes applying/dispensing an indicative substance on a target object and/or surface, whereafter, in step 320, light is transmitted to the object and/or the surface, so as to induce the indicative substance to emit luminescence. In step 330, the wavelength and/or intensity of the luminescence is detected using a detector, thereby determining a pre-treatment reference luminescence. In step 340 the object and/or surface is treated with plasma, which brings about a change in the indicative substance (e.g. in wavelength and/or intensity). In step 350, light is once again transmitted from the light source, and the post-treatment emission obtained from the indicative substance is detected in step 360. In step 370, the efficiency of the plasma treatment is determined based on the wavelength and/or intensity of the detected luminescence and its correlation with microorganism extermination and/or deactivation, as essentially described herein. Optionally, in step 380, the method may further include determining a time required and/or remaining for completion of the sterilization process. As a further option, in step 390, the efficiency of the plasma treatment, the required and/or remaining for completion of the sterilization process or any other suitable parameter of the sterilization process may be displayed on a display and/or graphic user interphase.

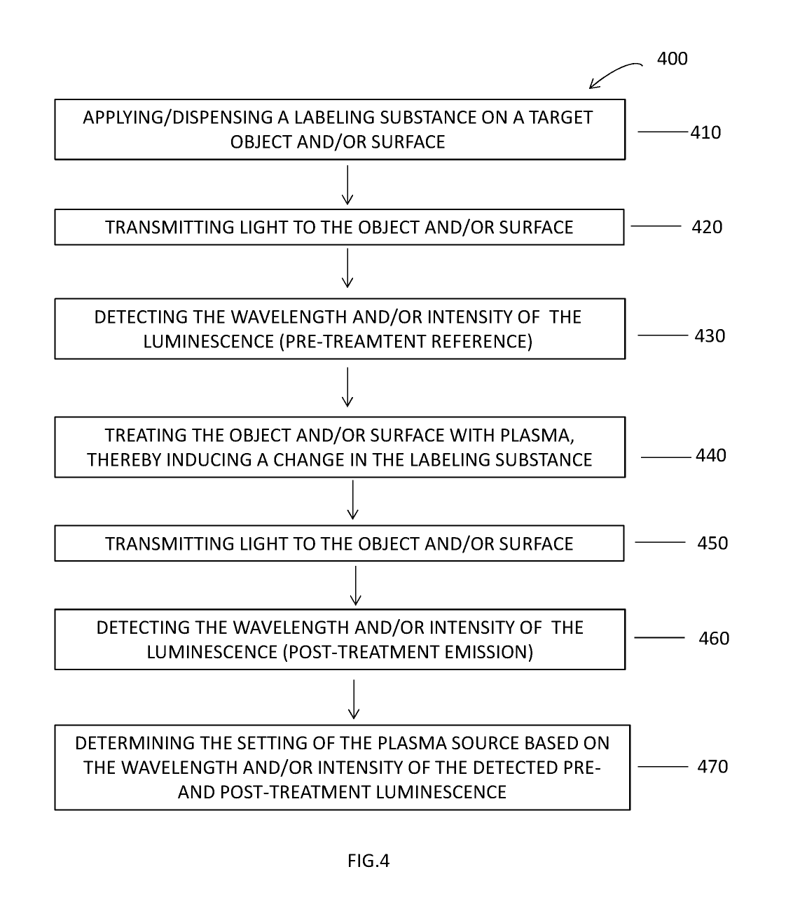

Reference is now made to FIG. 4, which is an exemplary flowchart for the operation of a plasma sterilization feedback processing system 400, according to some embodiments. Step 410 of the methods includes applying/dispensing an indicative substance on a target object and/or surface, whereafter, in step 420, light is transmitted to the object and/or the surface, so as to induce the indicative substance to emit luminescence. In step 430, the wavelength and/or intensity of the luminescence is detected using a detector, thereby determining a pre-treatment reference luminescence. In step 440 the object and/or surface is treated with plasma which brings about a change in the indicative substance (e.g. in wavelength and/or intensity). In step 450, light is once again transmitted from the light source, and the post-treatment emission obtained from the indicative substance is detected in step 460. In step 470, one or more settings of the plasma generator and/or plasma driver may be determined based on the wavelength and/or intensity of the detected luminescence, as essentially described herein. It is understood that the method may include additional steps such as, for example, determining the efficacy of the plasma treatment and/or the required/remaining for completion of the sterilization process may also be included in the method. Additionally or alternatively, the determination of the operational settings of the plasma generator may be based on the determined efficacy of the plasma treatment and/or its correlation with microorganism extermination and/or deactivation.

According to some embodiments, the plasma sterilizers (which may also be referred to herein as the plasma based decontamination instruments) disclosed herein, may be augmented with the real-time, on-line feedback system as described above.

One of the problems that may be encountered is that the area bombarded by the plasma beam may include a local sub-area where a concentration of pathogens remains high (for example a tiny hole or crevice). Its effect on the entire integrated luminosity as detected by the feedback system may be marginal or even beyond the system's sensitivity to detect. Nevertheless, it may form a serious danger to the patients as proven by many cases in the medical world when surgical or analytical tools that were thoroughly decontaminated according to the manufacturer's instructions, still retained a high concentration of potent pathogen in a small crevice, which later affected many people.

According to some embodiments, there is thus provided herein, a system configured to apply a selective concentration of decontamination area and feedback analyses area. According to some embodiments, the entire “bombarded” area may be divided into pixel-like sub-areas; each can be controlled individually as on/off operation of the plasma bombardment, as well as on/off analysis of the returned integrated luminosity from this specific pixel.

According to some embodiments, the system's integrated microprocessor can be commanded to ignore certain areas, at the operator's command, and analyze and bombard with plasma only chosen selected areas. According to some embodiments, the system's integrated microprocessor can be commanded to apply certain plasma characteristics on certain areas and analyze these selected areas accordingly, while applying other plasma characteristics on other selected areas and analyze these other selected areas accordingly.

Increased performance and sensitivity may thus be obtained, according to some embodiments, by dividing a bombarded area into many sub-areas, and controlling both the analysis of reading the emitted integrated fluorescent response from a pixel or pixel-like area, as well as whether to continue or to stop the bombardment of the plasma beam on a particular area.

According to some embodiments, this operation may be manually controlled on-line, or pre-programmed into the system's microprocessor. The system may automatically track the location of “hidden” or local contaminations or may be manually operated to position the concentration of the plasma bombardment.

According to some embodiments, an automatic tracking capability may be applied which allows the system to shift the concentration of analysis of the returned integrated light as well as the concentration of plasma bombardment to any specific residual hot-spot contaminated area which was discovered by the system. This feature may greatly enhance the capability of the feedback system and its associated plasma decontamination instrument, to eliminate the danger of leaving behind even a very small area which still was not properly decontaminated.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A plasma sterilizer comprising:
a plasma driver configured to provide plasma to a target area;
a dispenser configured to apply an indicative substance to the target area;
a light detector configured to detect an integrated luminescence intensity of the indicative substance from the target area; and
a feedback processing unit configured to obtain from said light detector information indicative of the emitted luminescence and to control the operation of said plasma driver based at least on the information.

2. The sterilizer of claim 1, further comprising a light source configured to provide light at a wavelength capable of inducing luminescence from said indicative substance.

3. The sterilizer of claim 1, further comprising a gas blender.

4. The sterilizer of claim 1, wherein controlling the operation of the plasma driver comprises controlling current, frequency, voltage, timing and modulation or any combination thereof.

5. The sterilizer of claim 1, wherein said sterilizer further comprises at least one gas supply input configured to provide said plasma generator with gas composition, wherein said gas composition is determined by said feedback processing unit and/or by manual input.

6. The sterilizer of claim 1, wherein said sterilizer further comprises one or more sensors configured to provide sensor signals to said feedback processing unit, wherein said feedback processing unit is configured to determine said gas composition and to determine the output setting of said plasma driver based on said sensor electric signals.

7. The sterilizer of claim 1, wherein the sensor signals comprise: in-flow gas composition, distance between the plasma generator and the target area, volume of confined space, ambient temperature, temperature on target surface, ambient humidity, current leakage from the plasma to the target, specific particle concentration or any combination thereof.

8. The sterilizer of claim 7, wherein the specific particle comprises NxOy or ozone.

9. The sterilizer of claim 1, wherein said light detector is configured to detect a wavelength of the emitted luminescence from the target area.

10. The sterilizer of claim 9, wherein said feedback processing unit is further configured to associate the wavelength and/or integrated intensity of the emitted luminescence from the targeted area, with an amount of plasma delivered to the target area and/or with a sterilization level achieved.

11. The sterilizer of claim 1, wherein said light detector is a camera.

12. The sterilizer of claim 1, wherein said indicative substance emits luminescence at a first wavelength before interaction with the plasma provided by said plasma generator and emits luminescence at a second wavelength after interaction with the plasma provided by said plasma generator.

13. The sterilizer of claim 12, wherein said light detector is configured to differentiate between said first and said second wavelength and/or associated integrated intensity.

14. The sterilizer of claim 12, wherein said feedback processing unit is configured to control the level and mode of activation of the plasma power driver and/or gas blender based on a ratio between said first and said second wavelengths and/or associated integrated intensity.

15. The sterilizer of claim 1, wherein the light source is a UV light source and wherein said luminescence comprises fluorescence, phosphorescence or both.

16. (canceled)

17. The sterilizer of claim 1, wherein said indicative substance is able to selectively bind and/or be absorbed by microorganisms.

18. The sterilizer of claim 1, further comprising at least one gas supply input, wherein the opening and/or closing and/or flow control of said gas supply input is controlled by said feedback processing unit.

19. The sterilizer of claim 18, wherein the at least one gas supply input comprises at least two gas supply inputs, wherein the opening and/or closing and/or flow control of each of said gas supply inputs is separately controlled by said feedback processing unit.

20. (canceled)

21. A method for plasma sterilizing, the method comprising:
applying an indicative substance to a target area;
utilizing a plasma driver, providing plasma to the target area;
detecting an integrated luminescence intensity of the indicative substance from the target area; and
utilizing a feedback processing unit, obtaining information indicative of the luminescence emitted from the target area and controlling one or more operational functions of the plasma driver based on an analysis of the luminescence emitted from the target area, wherein the luminescence emitted from the target area is indicative of the level of decontamination of target pathogen(s) on the target area.

22. A method for plasma sterilizing comprising:
applying an indicative substance on the targeted area, wherein the indicative substance comprises a “good bacteria” bound to or otherwise marked with a luminescence emitting substance, wherein the luminescence emitting substance is configured to emit luminescence only when it is bound to the “good bacteria” and/or only when the “good bacteria” is intact;
detecting an integrated intensity of the luminescence over the targeted area using a detector;
treating the targeted area with plasma using a plasma sterilizer, thereby inducing a change in the indicative substance; and
detecting the integrated intensity of the luminescence over the targeted area, thereby determining the level of sterilization of the target area.

23.-26. (canceled)
